(19) United States
(12) Patent Application Publication
(10) Pub. No.: US 2007/0148099 A1
(43) Pub. Date: Jun. 28, 2007

(54) Use of aroma compounds as defoaming agents for ophthalmic solutions with high concentrations of surfactants

(76) Inventors: Susan E. Burke, Batavia, NY (US); Daniel M. Ammon, Penfield, NY (US); Roya N. Borazjani, Ft. Worth, TX (US); Joseph C. Salamone, Boca Raton, FL (US); Erning Xia, Penfield, NY (US)

Correspondence Address: Bausch & Lomb Incorporated, One Bausch & Lomb Place, Rochester, NY, 14604-2701, US

(21) Appl. No.: 11/611,331

(22) Filed: Dec. 15, 2006

Related U.S. Application Data
(60) Provisional application No. 60/754,059, filed on Dec. 27, 2005.

(51) Int. Cl. A61K 9/12; A61K 31/045; A61K 31/05; A61K 31/075

(52) U.S. Cl. 424/45; 514/729; 514/731; 514/717

(57) ABSTRACT

A method of reducing foam formation and improving odor in ophthalmic solutions, such as multipurpose cleaning and conditioning solutions and contact lens packing solutions. The ophthalmic solutions contain surfactant and aroma compound in an amount effective to decrease foam formation. The aroma compound has a low molecular weight and pleasing smell.

### CROSS REFERENCE

This application claims the benefit of Provisional Application No. 60/754,059 filed on Dec. 27, 2005 and is incorporated herein by reference.

### FIELD

The present invention relates to ophthalmic compositions with reduced foam formation. In addition to reduced foaming, the ophthalmic compositions of the present invention have improved odor.

### BACKGROUND

Contact lenses are generally classified as hard or soft and as either water-absorptive or water-non-absorptive. Hard or rigid corneal lenses are formed from materials prepared by the polymerization of acrylic esters, such as poly(methyl methacrylate) (PMMA). Gel, hydrogel or soft-type lenses are made by polymerizing such monomers as 2-hydroxyethyl methacrylate (HEMA) or, in the case of extended wear lenses, by polymerizing silicon-containing monomers or macromonomers. Both the hard and soft types of contact lenses are exposed to a broad spectrum of microbes during normal wear and become soiled relatively quickly. Contact lenses, whether hard or soft, therefore require routine cleaning and disinfecting. Both hard and soft contact lenses may develop deposits or a stain of lipids derived from tears while the lens is worn. Such lipid stains may cause deterioration in the comfort of a lens during wear or cause eye problems such as blurred eyesight or congestion of the cornea. As such, it is essential to apply a cleaning treatment to a contact lens in order to safely and comfortably use contact lenses.

To effectively clean contact lenses, lens care solutions typically include a surfactant useful as a cleaning component. Contact lens cleaning solutions incorporating non-ionic surfactants such as a polyoxyalkylene block copolymer, including poly(ethylene oxide)-poly(propylene oxide) block copolymer or a derivative thereof, are known.

Great importance is attached to the safety and comfort of lens care solutions. Experience has shown that conventional lens care solutions containing cleaning surfactants at low concentrations lack adequate cleaning power or lipid-solubilizing power and tend to allow lipid stains to remain and accumulate on the contact lens, potentially harming the eye.

While desirable from the standpoint of product efficacy, the use of surfactants in lens care solutions can result in excessive foam generation, presenting problems during both manufacture and consumer use. In evaluation of lens care solutions, it has been shown that increasing the concentration of surfactants produces an increase in measured foam height. U.S. Patent Publication No. 2002/0141899 proposes a composition for disinfecting contact lenses with hydrogen peroxide and a surfactant comprising a block copolymer said to have a Ross Miles foam height of less than 1 mm.

U.S. Pat. No. 5,746,972 proposes a lens care composition comprising hydrogen peroxide and a surfactant. The composition is said to generate a reduced amount of foam when subjected to the action of catalase to cause the destruction of the hydrogen peroxide.

U.S. Pat. No. 6,790,816 proposes reducing mixing speed during the preparation of a lens care solution containing Tetronic 1107 to reduce foaming. However, this mechanical method slows the manufacturing process.

Multipurpose solutions that clean and disinfect contact lenses potentially have an unacceptable amount of foaming because of the surfactant employed. Multipurpose solutions have been described in the literature, for example, U.S. Pat. No. 4,820,352 to Riedhammer et al. and U.S. Pat. No. 5,096,607 to Mowrey-McKee et al. disclose such solutions. More generally, contact lens solutions are disclosed in U.S. Pat. No. 5,356,555 to Huth et al., U.S. Pat. No. 5,401,431 to Nakagawa et al., U.S. Pat. No. 5,409,546 to Nakagawa et al., U.S. Pat. No. 5,449,442 to Yamada et al., U.S. Pat. No. 5,487,788 to Kamiya et al., U.S. Pat. No. 5,505,953 to Chowhan, U.S. Pat. No. 5,556,480 to Rontome et al., U.S. Pat. No. 5,607,908 to Potini et al., U.S. Pat. No. 5,630,884 to Huth, U.S. Pat. No. 5,648,074 to Park et al., U.S. Pat. No. 5,654,262 to Desai et al., U.S. Pat. No. 5,800,807 to Huth et al., U.S. Pat. No. 5,820,696 to Kimura et al., U.S. Pat. No. 5,858,937 to Richard et al., U.S. Pat. No. 5,922,279 to Spooner, U.S. Pat. No. 6,024,954 to Park et al., and U.S. Pat. No. 6,121,327 to Tsuzuki et al.

The manufacturing and packaging of ophthalmic lens also involve aqueous solutions comprising surfactants. U.S. Patent Publication No. 2003/0109390 proposes contact lens packing solutions comprising non-ionic surfactants. U.S. Patent Publication No. 2004/0119176 proposes a method for manufacturing lenses with an aqueous solution comprising a surfactant, buffering agent and/or sodium chloride

Many commercial ophthalmic products, including certain lens care solutions, have also been developed without sufficient consideration to their sometimes less than pleasing appearance and odors.

U.S. Patent Publication Nos. 2005/0106271 and 2004/0052877 propose eye drops with clove, peppermint and ginger perfume.

JP No. 3197921 proposes a detergent for contact lens solutions wherein perfume is added to prevent organic odor.

JP No. 2001253822 proposes a corneal cell restore composition that includes a refreshing cooling agent such as menthol.

JP No. 2004143158, JP No. 2002128671, JP No. 2002104971, JP No. 2001097865, JP No. 9143064 and JP No. 7118147 all propose ophthalmic compositions that include menthol.

Accordingly, it would be desirable to add defoaming agents to aqueous lens care solutions containing surfactants.

It would also be desirable to provide improved defoaming for multipurpose lens care solutions having surfactants for cleaning and the enhancement of comfort.

It would also be desirable to provide improved defoaming for contact lens packing solutions because of the surfactants.

It would be further desirable to improve manufacturing processing time of ophthalmic solutions containing surfactants.

It would still further be desirable to improve the handling by consumers of ophthalmic solutions containing surfactants.

It would yet still further be desirable to develop lens care solutions having a generally pleasant odor.

### SUMMARY

In one aspect, the present invention relates to the use of aroma compounds in ophthalmic compositions, particularly lens care solutions, to reduce foaming. In another aspect, the present invention relates to the use of aroma compounds in ophthalmic solutions to improve unpleasant odors.

The invention therefore includes an aqueous composition for ophthalmic use comprising an effective defoaming amount of one or more aroma compounds.

The invention therefore further includes a method for reducing foam formation in an ophthalmic solution comprising adding an effective amount of one or more aroma compounds to reduce foaming.

### DETAILED DESCRIPTION

Multipurpose lens care solutions use one or more surfactants to reduce end of the day dryness, decrease protein and microbial buildup on lens surfaces and to prevent the attachment and/or up-take of disinfectants by the lens material. The concentration of surfactant can be limited by the potential for foaming. Elimination of foaming can expand possibilities for use of high foaming surfactants and combinations of surfactants. As is well known to those skilled in the art, foaming hinders the manufacturing process and handling of the lens care solution by the consumer.

Foam is a dispersion of a gas, such as air, in a liquid. The gas bubbles are separated by thin liquid films called lamellae, which are stabilized by the presence of a surfactant in solution. Foam properties are dependent on the type and concentration of surfactant.

The surfactant component of the lens care solutions, packaging solution or ophthalmic solution disclosed herein is present in an amount effective to remove deposit material from a contact lens in the liquid medium. Generally, the concentration of surfactant is a minimum of about 0.1% by weight, about 0.3% by weight or about 0.5% by weight and or a maximum of about 6.0% by weight, about 2.0% by weight or 1% by weight. Preferably, the concentration of the surfactant is about 1.0% by weight.

Generally, useful surfactants are nonionic, water-soluble surfactants. Generally, the surfactants will have a hydrophilic-lipophilic balance (HLB) greater than about 8 and a molecular weight in the range of 400 to 30,000.

One class of preferred surfactant is block copolymers of ethylene oxide and propylene oxide, where the ratio of poly(ethylene oxide) and poly(propylene oxide) repeating units determines the hydrophilic-lipophilic balance of the surfactant. As a first example, poloxamers are poly(ethylene oxide), poly(propylene oxide) block polymers available under the tradename Pluronic® (BASF Wyandotte Corp., Wyandotte, Mich.). Specific poloxamers include poloxamer 407 (available as Pluronic® F-127) and poloxamer 108 (available as Pluronic® F-38). An additional example is meroxapol 105 (available as Pluronic® 10 R5). As a second example, poloxamines are ethylene diamine adducts of such poly(ethylene oxide), poly(propylene oxide) block polymers available under the tradename Tetronic® (BASF Wyandotte Corp.). Specific poloxamines include poloxamine 1107 (available as Tetronic® 1107) having a molecular weight from about 7,500 to about 27,000 wherein at least 40 weight percent of said adduct is poly(ethylene oxide), and poloxamine 1304 (available as Tetronic® 1304).

Particularly preferred surfactants include but are not limited to Pluronic® L10, L35, F38, L43, L44, L63, L64, P65, F68, F68LF, P75, F77, P84, P85, F87, F88, F98, P103, P104, P105, F108 and F127, as well as Tetronic® 304, 504, 704, 707, 904, 908, 909, 1104, 1107, 1304, 1307, 1504, and 1508.

Another class of surfactants is the various polyethylene glycol ethers of stearyl alcohol. A specific example is steareth-100, available under the tradename Brij® 700 (ICI Americas).

Other non-ionic surfactants include: polyethylene glycol esters of fatty acids, e.g. coconut, polysorbate, polyoxyethylene or polyoxypropylene ethers of higher alkanes (C₁₂-C₁₈); polysorbate 20 (available under the trademark Tween® 20); polyoxyethylene (23) lauryl ether (available under the tradename Brij® 35); polyoxyethyeneglycol (40) stearate (available under the tradename Myrj® 52); polyoxyethyeneglycol (20) stearate (available under the tradename Myrj® 49); and polyoxyethylene (25) propylene glycol stearate (available under the tradename Atlas® G 2612).

Various other surfactants suitable for use herein can be readily ascertained, in view of the foregoing description, from McCutcheon's Detergents and Emulsifiers, North American Edition, McCutcheon Division, MC Publishing Co., Glen Rock, N.J. 07452 and the CTFA International Cosmetic Ingredient Handbook, Published by The Cosmetic, Toiletry, and Fragrance Association, Washington, D.C.

As indicated hereinabove, some surfactants that are useful in ophthalmic solutions have unacceptable amount of foaming at certain concentrations. It is believed that the aroma chemicals disclosed herein destabilize the lamellae walls within the foam, thereby reducing the accumulation of bubbles at the air liquid interface.

In the process disclosed herein, an effective amount of one or more aroma compounds is added to the ophthalmic solution. In particular, low molecular weight aroma compounds are used. Generally, the molecular weight is less than about 300. The aroma compounds of the present invention are neutral and do not contain an ionic functional group. The aroma compounds are also soluble in the ophthalmic solution comprising surfactant.

Suitable examples of aroma compounds include, but are not limited to terpenes, terpenoids, and terpene-like compounds. Terpenes are a class of hydrocarbons produced by many living plants. They are derivatives of isoprene, with the isoprene units generally arranged in a head-to-toe fashion. Terpene derivatives that are saturated, partially saturated, or that contain functional groups such as alcohols, aldehydes, and esters are classified as terpenoids. (see “Kirk-Othmer Encyclopedia of Chemical Technology”, 3^{rd} ed., 1978, Vol. 22, pages 709-762, Grayson, M.; Eckroth, D. eds.). Some examples of terpenes, terpenoids, and terpene-like compounds include: tricyclene, α-pinene, limonene, terpinolene, carveol, citronellol, amyl alcohol, camphor, nerol, β-santalol, eugenol, α-terpineol, caryophyllene, guaiol, anisaldehyde, cedrol, linalool, longifolene, anisyl alcohol, patchouli alcohol, α-cadinene, menthol, 1,8-cineole, p-cymene, 3-carene, p-8-menthane, trans-menthone, bomeol, α-fenchol, isoamyl acetate, terpin, citronellal, geraniol, 1-terpinen-4-ol, and famesol. The aroma compound is generally added in a concentration in the range of from about 0.025 ppm to about 30.0 ppm; particularly in the range of from about 0.025 ppm to about 3.0 ppm; more particularly in the range of from about 0.05 ppm to about 1.0 ppm; and most particularly in the range of from about 0.1 ppm to about 0.6 ppm.

The aroma compounds described herein have an added advantage of improving the odor of ophthalmic solutions. As is well known, odor is the sensation caused by those properties of any substance entering into the mouth that stimulates simultaneously both olfactory and trigeminal systems. An odor is a sensation of substances that may be substantially provided by a single chemical entity or a blend of chemicals of natural or synthetic origin. Opthalmic solutions containing the aroma compounds of the present invention generally have a mild odor.

Optionally, the ophthalmic solutions of the present invention may include a buffering agent, a tonicity agent, a sequestering agent, a wetting agent, a conditioning agent, and/or an antimicrobial agent.

### EXAMPLES

The following non-limiting examples illustrate certain aspects of the present invention.

### Example 1

A multipurpose lens care solution is prepared having the composition shown in Table 1 below.

TABLE 1

| | Lens Care Solution |
|---|---|
| Compound | Concentration (% w/w) |
| Boric Acid | 0.85 |
| Sodium Phosphate | 0.15 |
| (Monobasic) | |
| Sodium Phosphate | 0.31 |
| (Dibasic) | |
| Sodium Chloride | 0.06 |
| Hydroxyalkyl | 0.1 |
| Phosphonate (30%) | |
| Poloxamer 407 | 3.0 |
| (Surfactant) | |
| Poloxamine 1107 | 1.5 |
| (Surfactant) | |
| Polyquaternium 10 | 0.02 |
| Alexidine | 4.5 ppm |
| Purified Water | 100% Q. S. |

### Example 2

Varying amounts of α-terpineol are added to the multipurpose lens care solution of Example 1. The foam height is measured after vigorous shaking of a 100 ml graduated cylinder containing 50 ml of the solution (0 minutes elapsed time) and after 30 minutes of standing time. The results are set forth in Table 2 below. Within the concentration range investigated, the multipurpose lens care solution has a mild odor. The odor description is based upon the perception of the scientist conducting the experiment.

Table 2 shows a decrease in the time required for foam draining in the presence of α-terpineol.

TABLE 2

| | Foam Height (cm) | Foam Height (cm) |
|---|---|---|
| α-Terpineol (ppm) | Time = 0 minutes | Time = 30 minutes |
| 0 | 7.1 ± 0.3 | 6.8 ± 0.4 |
| 0.025 | 5.7 ± 0.3 | 5.0 ± 0.3 |
| 0.05 | 4.8 ± 0.2 | 3.2 ± 0.3 |
| 0.10 | 3.7 ± 0.2 | 0.3 ± 0.1 |
| 0.20 | 2.2 ± 0.1 | 0.2 ± 0.1 |
| 0.30 | 1.1 ± 0.2 | 0.2 ± 0.1 |
| 0.40 | 1.0 ± 0.1 | 0.1 ± 0.0 |
| 0.50 | 0.8 ± 0.1 | 0.0 ± 0.0 |
| 0.60 | 0.6 ± 0.1 | 0.0 ± 0.0 |
| 0.70 | 0.6 ± 0.1 | 0.0 ± 0.0 |
| 0.80 | 0.6 ± 0.1 | 0.0 ± 0.0 |
| 0.90 | 0.6 ± 0.1 | 0.0 ± 0.0 |
| 1.00 | 0.6 ± 0.1 | 0.0 ± 0.0 |
| 2.50 | 0.5 ± 0.1 | 0.0 ± 0.0 |

### Example 3

Varying amounts of geraniol are added to the multipurpose lens care solution of Example 1. The foam height is measured after vigorous shaking of a 100 ml graduated cylinder containing 50 ml of the solution (0 minutes elapsed time) and after 30 minutes of standing time. The results are set forth in Table 3 below. Within the concentration range investigated, the multipurpose lens care solution has a mild odor. The odor description is based upon the perception of the scientist conducting the experiment.

Table 3 shows a decrease in the time required for foam draining in the presence of geraniol.

TABLE 3

| | Foam Height (cm) | Foam Height (cm) |
|---|---|---|
| Geraniol (ppm) | Time = 0 minutes | Time = 30 minutes |
| 0 | 7.1 ± 0.3 | 6.8 ± 0.4 |
| 0.025 | 3.0 ± 0.3 | 0.8 ± 0.3 |
| 0.05 | 1.3 ± 0.3 | 0.2 ± 0.1 |
| 0.10 | 0.6 ± 0.3 | 0.0 ± 0.0 |
| 0.20 | 0.4 ± 0.2 | 0.0 ± 0.0 |
| 0.30 | 0.3 ± 0.1 | 0.0 ± 0.0 |
| 0.40 | 0.3 ± 0.2 | 0.0 ± 0.0 |
| 0.50 | 0.2 ± 0.1 | 0.0 ± 0.0 |
| 0.60 | 0.1 ± 0.0 | 0.0 ± 0.0 |
| 0.70 | 0.1 ± 0.1 | 0.0 ± 0.0 |
| 0.80 | 0.1 ± 0.1 | 0.0 ± 0.0 |
| 0.90 | 0.1 ± 0.1 | 0.0 ± 0.0 |
| 1.00 | 0.1 ± 0.1 | 0.0 ± 0.0 |
| 2.50 | 0.1 ± 0.1 | 0.0 ± 0.0 |

### Example 4

Varying amounts of anisyl alcohol are added to the multipurpose lens care solution of Example 1. The foam height is measured after vigorous shaking of a 100 ml graduated cylinder containing 50 ml of the solution (0 minutes elapsed time) and after 30 minutes of standing time. The results are set forth in Table 4 below. Within the concentration range investigated, the multipurpose lens care solution has a mild odor. The odor description is based upon the perception of the scientist conducting the experiment.

Table 4 shows a decrease in the time required for foam draining in the presence of anisyl alcohol.

TABLE 4

| Anisyl Alcohol | Foam Height (cm) | Foam Height (cm) |
|---|---|---|
| (ppm) | Time = 0 min | Time = 30 min |
| 0 | 7.3 ± 0.4 | 6.8 ± 0.4 |
| 0.025 | 3.6 ± 0.4 | 3.2 ± 0.4 |
| 0.05 | 3.2 ± 0.3 | 2.4 ± 0.2 |
| 0.10 | 2.8 ± 0.2 | 1.9 ± 0.3 |
| 0.20 | 2.4 ± 0.1 | 1.5 ± 0.2 |
| 0.30 | 2.0 ± 0.1 | 1.3 ± 0.3 |
| 0.40 | 1.7 ± 0.2 | 1.2 ± 0.2 |
| 0.50 | 1.5 ± 0.3 | 1.1 ± 0.3 |
| 0.60 | 1.4 ± 0.3 | 1.1 ± 0.2 |
| 0.70 | 1.3 ± 0.2 | 1.0 ± 0.2 |
| 0.80 | 1.2 ± 0.2 | 0.9 ± 0.2 |
| 0.90 | 1.0 ± 0.1 | 0.8 ± 0.2 |
| 1.00 | 0.9 ± 0.2 | 0.5 ± 0.1 |
| 2.50 | 0.4 ± 0.1 | 0.3 ± 0.1 |

### Example 5

Varying amounts of carveol are added to the multipurpose lens care solution of Example 1. The foam height is measured after vigorous shaking of a 100 ml graduated cylinder containing 50 ml of the solution (0 minutes elapsed time) and after 30 minutes of standing time. The results are set forth in Table 5 below. Within the concentration range investigated, the multipurpose lens care solution has a mild odor. The odor description is based upon the perception of the scientist conducting the experiment.

Table 5 shows a decrease in the time required for foam draining in the presence of carveol.

TABLE 5

| | Foam Height (cm) | Foam Height (cm) |
|---|---|---|
| Carveol (ppm) | Time = 0 min | Time = 30 min |
| 0 | 7.3 ± 0.4 | 6.8 ± 0.4 |
| 0.025 | 5.8 ± 0.4 | 4.8 ± 0.3 |
| 0.05 | 5.4 ± 0.2 | 3.6 ± 0.3 |
| 0.10 | 4.7 ± 0.3 | 2.4 ± 0.2 |
| 0.20 | 3.8 ± 0.2 | 1.8 ± 0.3 |
| 0.30 | 3.0 ± 0.3 | 1.4 ± 0.2 |
| 0.40 | 2.6 ± 0.3 | 1.2 ± 0.2 |
| 0.50 | 2.1 ± 0.2 | 1.0 ± 0.2 |
| 0.60 | 2.0 ± 0.3 | 1.0 ± 0.2 |
| 0.70 | 1.9 ± 0.2 | 0.9 ± 0.1 |
| 0.80 | 1.8 ± 0.2 | 0.9 ± 0.2 |
| 0.90 | 1.6 ± 0.1 | 0.7 ± 0.1 |
| 1.00 | 1.1 ± 0.2 | 0.6 ± 0.1 |
| 2.50 | 0.7 ± 0.1 | 0.5 ± 0.1 |

### Example 6

Varying amounts of 1,8-cineole are added to the multipurpose lens care solution of Example 1. The foam height is measured after vigorous shaking of a 100 ml graduated cylinder containing 50 ml of the solution (0 minutes elapsed time) and after 30 minutes of standing time. The results are set forth in Table 6 below. Within the concentration range investigated, the multipurpose lens care solution has a mild odor. The odor description is based upon the perception of the scientist conducting the experiment.

Table 6 shows a decrease in the time required for foam draining in the presence of 1,8-cineole.

TABLE 6

| | Foam Height (cm) | Foam Height (cm) |
|---|---|---|
| 1,8-Cineole (ppm) | Time = 0 min | Time = 30 min |
| 0 | 7.3 ± 0.4 | 6.8 ± 0.4 |
| 0.025 | 6.3 ± 0.3 | 4.5 ± 0.3 |
| 0.05 | 5.8 ± 0.2 | 3.7 ± 0.3 |
| 0.10 | 5.5 ± 0.3 | 3.2 ± 0.2 |
| 0.20 | 5.0 ± 0.3 | 2.9 ± 0.2 |
| 0.30 | 4.3 ± 0.1 | 2.7 ± 0.1 |
| 0.40 | 3.5 ± 0.2 | 2.5 ± 0.2 |
| 0.50 | 3.1 ± 0.3 | 2.4 ± 0.1 |
| 0.60 | 3.0 ± 0.1 | 2.3 ± 0.2 |
| 0.70 | 2.8 ± 0.1 | 2.2 ± 0.3 |
| 0.80 | 2.7 ± 0.2 | 2.1 ± 0.2 |
| 0.90 | 2.4 ± 0.3 | 1.8 ± 0.2 |
| 1.00 | 2.0 ± 0.2 | 1.5 ± 0.1 |
| 2.50 | 1.6 ± 0.1 | 1.3 ± 0.1 |

### Example 7

Varying amounts of eugenol are added to the multipurpose lens care solution of Example 1. The foam height is measured after vigorous shaking of a 100 ml graduated cylinder containing 50 ml of the solution (0 minutes elapsed time) and after 30 minutes of standing time. The results are set forth in Table 7 below. Within the concentration range investigated, the multipurpose lens care solution has a mild odor. The odor description is based upon the perception of the scientist conducting the experiment.

Table 7 shows a decrease in the time required for foam draining in the presence of eugenol.

TABLE 7

| | Foam Height (cm) | Foam Height (cm) |
|---|---|---|
| Eugenol (ppm) | Time = 0 min | Time = 30 min |
| 0 | 7.3 ± 0.4 | 6.8 ± 0.4 |
| 0.025 | 2.7 ± 0.2 | 1.6 ± 0.2 |
| 0.05 | 1.8 ± 0.2 | 1.0 ± 0.2 |
| 0.10 | 1.0 ± 0.2 | 0.6 ± 0.2 |
| 0.20 | 0.8 ± 0.2 | 0.5 ± 0.3 |
| 0.30 | 0.6 ± 0.2 | 0.4 ± 0.2 |
| 0.40 | 0.5 ± 0.2 | 0.3 ± 0.2 |
| 0.50 | 0.4 ± 0.2 | 0.2 ± 0.1 |
| 0.60 | 0.3 ± 0.2 | 0.1 ± 0.1 |
| 0.70 | 0.2 ± 0.2 | 0.1 ± 0.1 |
| 0.80 | 0.2 ± 0.1 | 0.1 ± 0.1 |
| 0.90 | 0.2 ± 0.1 | 0.1 ± 0.1 |
| 1.00 | 0.2 ± 0.1 | 0.1 ± 0.1 |
| 2.50 | 0.2 ± 0.1 | 0.1 ± 0.1 |

### Example 8

Varying amounts of amyl alcohol are added to the multipurpose lens care solution of Example 1. The foam height is measured after vigorous shaking of a 100 ml graduated cylinder containing 50 ml of the solution (0 minutes elapsed time) and after 30 minutes of standing time. The results are set forth in Table 8 below. Within the concentration range investigated, the multipurpose lens care solution has a mild odor. The odor description is based upon the perception of the scientist conducting the experiment.

Table 8 shows a decrease in the time required for foam draining in the presence of amyl alcohol.

TABLE 8

| Amyl Alcohol | Foam Height (cm) | Foam Height (cm) |
|---|---|---|
| (ppm) | Time = 0 min | Time = 30 min |
| 0 | 7.8 ± 0.3 | 6.5 ± 0.3 |
| 0.025 | 7.6 ± 0.3 | 5.5 ± 0.3 |
| 0.05 | 7.5 ± 0.2 | 5.2 ± 0.2 |
| 0.10 | 6.7 ± 0.2 | 5.0 ± 0.3 |
| 0.20 | 6.2 ± 0.3 | 4.7 ± 0.3 |
| 0.30 | 4.8 ± 0.2 | 3.7 ± 0.2 |
| 0.40 | 3.9 ± 0.1 | 3.0 ± 0.1 |
| 0.50 | 3.3 ± 0.2 | 2.2 ± 0.2 |
| 0.60 | 2.1 ± 0.3 | 1.4 ± 0.3 |
| 0.70 | 1.8 ± 0.1 | 1.2 ± 0.1 |
| 0.80 | 1.5 ± 0.1 | 1.0 ± 0.1 |
| 0.90 | 1.0 ± 0.1 | 0.8 ± 0.1 |
| 1.00 | 0.6 ± 0.1 | 0.4 ± 0.1 |
| 2.50 | 0.4 ± 0.1 | 0.3 ± 0.1 |

### Example 9

Varying amounts of isoamyl acetate are added to the multipurpose lens care solution of Example 1. The foam height is measured after vigorous shaking of a 100 ml graduated cylinder containing 50 ml of the solution (0 minutes elapsed time) and after 30 minutes of standing time. The results are set forth in Table 9 below. Within the concentration range investigated, the multipurpose lens care solution has a mild odor. The odor description is based upon the perception of the scientist conducting the experiment.

Table 9 shows a decrease in the time required for foam draining in the presence of isoamyl acetate.

TABLE 9

| Isoamyl Acetate | Foam Height (cm) | Foam Height (cm) |
|---|---|---|
| (ppm) | T = 0 | T = 30 min |
| 0 | 7.2 ± 0.3 | 7.0 ± 0.4 |
| 0.025 | 5.9 ± 0.2 | 4.5 ± 0.3 |
| 0.05 | 4.6 ± 0.3 | 3.6 ± 0.1 |
| 0.10 | 4.2 ± 0.1 | 3.3 ± 0.2 |
| 0.20 | 3.6 ± 0.3 | 3.0 ± 0.3 |
| 0.30 | 3.3 ± 0.2 | 2.7 ± 0.1 |
| 0.40 | 3.1 ± 0.1 | 2.5 ± 0.2 |
| 0.50 | 3.0 ± 0.2 | 2.5 ± 0.2 |
| 0.60 | 3.0 ± 0.3 | 2.3 ± 0.2 |
| 0.70 | 2.7 ± 0.4 | 2.2 ± 0.1 |
| 0.80 | 2.6 ± 0.1 | 2.1 ± 0.3 |
| 0.90 | 2.5 ± 0.3 | 2.0 ± 0.1 |
| 1.00 | 2.2 ± 0.2 | 1.9 ± 0.2 |
| 2.50 | 2.1 ± 0.1 | 1.8 ± 0.1 |

### Example 10

Varying amounts of linalool are added to the multipurpose lens care solution of Example 1. The foam height is measured after vigorous shaking of a 100 ml graduated cylinder containing 50 ml of the solution (0 minutes elapsed time) and after 30 minutes of standing time. The results are set forth in Table 10 below. Within the concentration range investigated, the multipurpose lens care solution has a mild odor. The odor description is based upon the perception of the scientist conducting the experiment.

Table 10 shows a decrease in the time required for foam draining in the presence of linalool.

TABLE 10

| | Foam Height (cm) | Foam Height (cm) |
|---|---|---|
| Linalool (ppm) | Time = 0 min | Time = 30 min |
| 0 | 7.3 ± 0.4 | 6.8 ± 0.4 |
| 0.025 | 5.3 ± 0.3 | 4.7 ± 0.3 |
| 0.05 | 4.6 ± 0.2 | 4.0 ± 0.2 |
| 0.10 | 3.7 ± 0.1 | 2.6 ± 0.3 |
| 0.20 | 2.5 ± 0.1 | 2.0 ± 0.2 |
| 0.30 | 1.8 ± 0.2 | 1.1 ± 0.3 |
| 0.40 | 1.1 ± 0.2 | 0.7 ± 0.3 |
| 0.50 | 0.6 ± 0.3 | 0.5 ± 0.1 |
| 0.60 | 0.6 ± 0.1 | 0.4 ± 0.2 |
| 0.70 | 0.5 ± 0.2 | 0.4 ± 0.1 |
| 0.80 | 0.5 ± 0.3 | 0.3 ± 0.2 |
| 0.90 | 0.4 ± 0.1 | 0.2 ± 0.1 |
| 1.00 | 0.3 ± 0.1 | 0.1 ± 0.1 |
| 2.50 | 0.2 ± 0.1 | 0.1 ± 0.1 |

### Example 11

Varying amounts of anisaldehyde are added to the multipurpose lens care solution of Example 1. The foam height is measured after vigorous shaking of a 100 ml graduated cylinder containing 50 ml of the solution (0 minutes elapsed time) and after 30 minutes of standing time. The results are set forth in Table 11 below. Within the concentration range investigated, the multipurpose lens care solution has a mild odor. The odor description is based upon the perception of the scientist conducting the experiment.

Table 11 shows a decrease in the time required for foam draining in the presence of anisaldehyde.

TABLE 11

| Anisaldehyde | Foam Height (cm) | Foam Height (cm) |
|---|---|---|
| (ppm) | Time = 0 min | Time = 30 min |
| 0 | 7.3 ± 0.4 | 6.8 ± 0.4 |
| 0.025 | 6.9 ± 0.3 | 6.0 ± 0.2 |
| 0.05 | 5.3 ± 0.3 | 4.4 ± 0.3 |
| 0.10 | 4.6 ± 0.4 | 3.6 ± 0.2 |
| 0.20 | 4.2 ± 0.2 | 3.4 ± 0.2 |
| 0.30 | 4.0 ± 0.2 | 3.1 ± 0.3 |
| 0.40 | 3.8 ± 0.2 | 2.8 ± 0.2 |
| 0.50 | 3.6 ± 0.1 | 2.6 ± 0.1 |
| 0.60 | 3.0 ± 0.1 | 2.2 ± 0.3 |
| 0.70 | 2.5 ± 0.2 | 1.9 ± 0.2 |
| 0.80 | 2.2 ± 0.2 | 1.6 ± 0.2 |
| 0.90 | 2.0 ± 0.2 | 1.5 ± 0.1 |
| 1.00 | 1.7 ± 0.1 | 1.4 ± 0.1 |
| 2.50 | 1.6 ± 0.2 | 1.3 ± 0.1 |

### Example 12

Varying amounts of 1-terpinen-4-ol are added to the multipurpose lens care solution of Example 1. The foam height is measured after vigorous shaking of a 100 ml graduated cylinder containing 50 ml of the solution (0 minutes elapsed time) and after 30 minutes of standing time. The results are set forth in Table 12 below. Within the concentration range investigated, the multipurpose lens care solution has a mild odor. The odor description is based upon the perception of the scientist conducting the experiment.

Table 12 shows a decrease in the time required for foam draining in the presence of 1-terpinen-4-ol.

TABLE 12

| 1-Terpinen-4-ol | Foam Height (cm) | Foam Height (cm) |
|---|---|---|
| (ppm) | Time = 0 min | Time = 30 min |
| 0 | 7.3 ± 0.4 | 6.8 ± 0.4 |
| 0.025 | 4.5 ± 0.2 | 3.7 ± 0.3 |
| 0.05 | 3.5 ± 0.3 | 2.6 ± 0.2 |
| 0.10 | 2.6 ± 0.2 | 2.0 ± 0.4 |
| 0.20 | 2.5 ± 0.2 | 1.8 ± 0.3 |
| 0.30 | 2.4 ± 0.1 | 1.6 ± 0.2 |
| 0.40 | 2.2 ± 0.2 | 1.5 ± 0.1 |
| 0.50 | 2.0 ± 0.2 | 1.4 ± 0.2 |
| 0.60 | 1.8 ± 0.3 | 1.3 ± 0.2 |
| 0.70 | 1.6 ± 0.1 | 1.2 ± 0.1 |
| 0.80 | 1.5 ± 0.2 | 1.1 ± 0.1 |
| 0.90 | 1.3 ± 0.2 | 0.9 ± 0.1 |
| 1.00 | 1.0 ± 0.2 | 0.7 ± 0.1 |
| 2.50 | 0.9 ± 0.2 | 0.5 ± 0.1 |

The use of the aroma compounds in Examples 2 through 12 as defoaming agents is applicable to aqueous lens care compositions, particularly multipurpose lens care solutions. The multipurpose lens care solution of Example 1 contained 4.5 weight % surfactants. Defoaming provides improved processing and handling of ophthalmic solutions. The aroma compounds also provide a pleasant odor to the multipurpose solution.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

What is claimed is:

1. An aqueous composition for ophthalmic use comprising an effective defoaming amount of one or more aroma compounds.

2. The composition of claim 1, wherein said one or more aroma compounds is soluble in said aqueous composition for ophthalmic use.

3. The composition of claim 1, wherein said one or more aroma compounds is neutral.

4. The composition of claim 1, wherein said one or more aroma compounds has a molecular weight of less than about 300.

5. The composition of claim 1, wherein said one or more aroma compounds is in an amount in the range of from about 0.025 to about 30.0 ppm.

6. The composition of claim 5, wherein said one or more aroma compounds is in an amount in the range of from about 0.025 to about 3.0 ppm.

7. The composition of claim 6, wherein said one or more aroma compounds is in an amount in the range of from about 0.05 to about 1.0 ppm.

8. The composition of claim 7, wherein said one or more aroma compounds is in an amount in the range of from about 0.1 to about 0.6 ppm.

9. The composition of claim 1, wherein the defoaming amount of one or more aroma compounds reduces foam height by at least about 5 cm.

10. The composition of claim 1, wherein said one or more aroma compounds is selected from the group consisting of terpenes, terpenoids, and/or terpene-like compounds.

11. The composition of claim 1, wherein said one or more aroma compounds is selected from the group consisting of c-terpineol, geraniol, menthol, camphor, amyl alcohol, isoamyl acetate, 1-terpinen-4-ol, 1,8-cineole, anisyl alcohol, linalool, anisaldehyde, eugenol, carveol, and/or mixtures thereof.

12. The composition of claim 11, wherein said one or more aroma compounds is geraniol.

13. The composition of claim 11, wherein said one or more aroma compounds is amyl alcohol.

14. The composition of claim 11, wherein said one or more aroma compounds is isoamyl acetate.

15. The composition of claim 11, wherein said one or more aroma compounds is 1-terpinen-4-ol.

16. The composition of claim 11, wherein said one or more aroma compounds is 1,8-cineole.

17. The composition of claim 11, wherein said one or more aroma compounds is anisyl alcohol.

18. The composition of claim 11, wherein said one or more aroma compounds is linalool.

19. The composition of claim 11, wherein said one or more aroma compounds is anisaldehyde.

20. The composition of claim 11, wherein said one or more aroma compounds is eugenol.

21. The composition of claim 11, wherein said one or more aroma compounds is carveol.

22. The composition of claim 1, further comprising one or more surfactants.

23. The composition of claim 22, wherein said one or more surfactants has an HLB value of greater than about 8.

24. The composition of claim 22, wherein said one or more surfactants is selected from the group consisting of poloxamers, poloxamines, and/or mixtures thereof.

25. The composition of claim 22, wherein said one or more surfactants is in a concentration in the range of from about 0.01% by weight to about 6.0% by weight based on total concentration of said aqueous composition for ophthalmic use.

26. The composition of claim 25, wherein said one or more surfactants is in a concentration in the range of about 0.05% by weight to about 2% by weight based on total concentration of said aqueous composition for ophthalmic use.

27. The composition of claim 1, having a composition foam height of less than about 5.0 cm measured after shaking at 22° C. and 0 minutes elapsed time.

28. The composition of claim 27, having a composition foam height of less than about 4.0 cm measured after shaking at 22° C. and 30 minutes elapsed time.

29. A method for reducing foam formation in an ophthalmic solution comprising adding an effective amount of one or more aroma compounds to reduce foaming.

30. The method of claim 29, wherein said one or more aroma compounds is soluble in said ophthalmic solution.

31. The method of claim 29, wherein said one or more aroma compounds is neutral.

32. The method of claim 29, wherein said one or more aroma compounds has a molecular weight of less than about 300.

33. The method of claim 29, wherein said one or more aroma compounds is in an amount in the range of from about 0.025 to about 30.0 ppm.

34. The method of claim 33, wherein said one or more aroma compounds is in an amount in the range of from about 0.025 to about 3.0 ppm.

35. The method of claim 34, wherein said one or more aroma compounds is in an amount in the range of from about 0.05 to about 1.0 ppm.

36. The method of claim 35, wherein said one or more aroma compounds is in an amount in the range of from about 0.1 to about 0.6 ppm.

37. The method of claim 29, wherein the foam formation is reduced by at least about 5 cm.

38. The method of claim 29, wherein said one or more aroma compounds is selected from the group consisting of terpenes, terpenoids, and/or terpene-like compounds.

39. The method of claim 29, wherein said one or more aroma compounds is selected from the group consisting of α-terpineol, geraniol, menthol, camphor, amyl alcohol, isoamyl acetate, 1-terpinen-4-ol, 1,8-cineole, anisyl alcohol, linalool, anisaldehyde, eugenol, carveol, and/or mixtures thereof.

40. The method of claim 39, wherein said one or more aroma compounds is geraniol.

41. The method of claim 39, wherein said one or more aroma compounds is amyl alcohol.

42. The method of claim 39, wherein said one or more aroma compounds is isoamyl acetate.

43. The method of claim 39, wherein said one or more aroma compounds is 1-terpinen-4-ol.

44. The method of claim 39, wherein said one or more aroma compounds is 1,8-cineole.

45. The method of claim 39, wherein said one or more aroma compounds is anisyl alcohol.

46. The method of claim 39, wherein said one or more aroma compounds is linalool.

47. The method of claim 39, wherein said one or more aroma compounds is anisaldehyde.

48. The method of claim 39, wherein said one or more aroma compounds is eugenol.

49. The method of claim 39, wherein said one or more aroma compounds is carveol.

50. The method of claim 29, wherein the opthalmic solution further comprises one or more surfactants.

51. The method of claim 50, wherein said one or more surfactants has an HLB value of greater than about 8.

52. The method of claim 50, wherein said one or more surfactants is selected from the group consisting of poloxamers, poloxamines, and/or mixtures thereof.

53. The method of claim 50, wherein said one or more surfactants is in a concentration in the range of from about 0.01% by weight to about 6.0% by weight based on total concentration of said ophthalmic solution.

54. The method of claim 53, wherein said one or more surfactants is in a concentration in the range of about 0.05% by weight to about 2.0% by weight based on total concentration of said ophthalmic solution.

55. The method of claim 29, having a solution foam height of less than about 5.0 cm measured after shaking at 22° C. and 0 minutes elapsed time.

56. The method of claim 55, having a solution foam height of less than about 4.0 cm measured after shaking at 22° C. and 30 minutes elapsed time.
